(19) United States
(12) Patent Application Publication
(10) Pub. No.: US 2019/0202695 A1
(43) Pub. Date: Jul. 4, 2019

(54) SYNTHESIS OF A MESOPOROUS THREE DIMENSIONAL CARBON NITRIDE DERIVED FROM CYANAMIDE AND ITS USE IN THE KNOEVENAGEL REACTION

(71) Applicant: SABIC Global Technologies B.V., Bergen op Zoom (NL)

(72) Inventors: Siddulu N. Talapaneni, Mawson Lakes (AU); Ajayan Vinu, Mawson Lakes (AU); Ugo Ravon, Thuwal (SA); Khalid Albahily, Thuwal (SA)

(21) Appl. No.: 16/327,361

(22) PCT Filed: Aug. 18, 2017
(86) PCT No.: PCT/IB2017/055014

§ 371 Date: Feb. 22, 2019

Related U.S. Application Data
(60) Provisional application No. 62/377,812, filed on Aug. 22, 2016.

(51) Int. Cl. C01B 21/06; B01J 37/00; B01J 37/08; B01J 37/04; B01J 37/06; B01J 27/24; B01J 35/10; B01J 20/02; B01J 20/28; B01J 20/30; C07C 253/30

(52) U.S. Cl. CPC C01B 21/0605; C01P 2006/16; B01J 37/082; B01J 37/04; B01J 37/009; B01J 37/06; B01J 27/24; B01J 35/1019; B01J 35/1042; B01J 35/1061; B01J 20/0259; B01J 20/28061; B01J 20/28073; B01J 20/28083; B01J 20/3057; B01J 20/3078; B01J 20/3071; C07C 253/30; C01P 2004/04; C01P 2004/03; C01P 2002/85; C01P 2002/82; C01P 2006/12; C01P 2006/14; B01J 37/0018

(57) ABSTRACT

Mesoporous graphitic carbon nitride (MGCN) materials and method of making said MGCN materials is described. The MGCN materials include a three dimensional cyanamide based carbon nitride matrix having tunable pore diameters, a pore volume between 0.40 and 0.80 cm³ g⁻¹, and a surface area of 195 to 300 m² gm⁻¹. The matrix comprises sheets of three dimensionally arranged s-heptazine (tri-s-triazine) units. The MGCN materials are used as catalysts in aldol condensation reactions, in particular Knoevenagel reactions. The mesoporous structure is obtained by means of a silica template like KIT-6, which is removed after polymerisation of the cyanamide monomers.

### CROSS REFERENCE TO RELATED APPLICATIONS

This application claims benefit to U.S. Provisional Patent Application No. 62/377,812 filed Aug. 22, 2016, which is incorporated herein in its entirety.

### BACKGROUND

### 1. Field of the Invention

The invention generally concerns a mesoporous graphitic carbon nitride (MGCN) material having high nitrogen content. In particular, the invention concerns a MGCN material that includes sheets of three dimensionally arranged s-heptazine units, and has a pore volume between 0.40 and 0.80 cm³ g⁻¹ and a surface area of 195 to 300 m² gm⁻¹.

### 2. Description of Related Art

Due to its multiple surface functionalities and basic sites, carbon nitrides (CN) materials can be used as a catalyst in synthetic chemistry, such as the Knoevenagel condensation. Based on this principle, many researchers have attempted to optimize the CN synthesis for use in the Knoevenagel condensation. Vinu et al. (J. Mat. & Chem., 2012, Vol 22, (19), pp. 9831-9840) describes the the synthesis of a well-ordered 3D mesoporous carbon nitride (MCN-6) having various textural parameters through a simple polymerization reaction of carbon tetrachloride and ethylenediamine by using the 3D double gyroid mesoporous silica KIT-6 having various pore diameters as the sacrificial hard template. Antonietti et al. (Cat. Sci. Tech., 2012, Vol 2, pp. 1005-1009) describes the basic catalytic properties of deprotonated mpg-C₃N₄ using cyanamide as the precursor on a nanosized silica template with no mention of tunable pore sizes. Xin Li et al. (Cat. Lett., 2013, Vol 143, (6), pp. 600-609) describes the synthesis of 3D mesostructured graphitic carbon nitride materials using mesocellular silica foam (MCF) as template and investigates the effects of the C:N ratios on physicochemical properties.

Many of the aforementioned catalysts suffer in that they have limited surface area and chemical reactivity due to lack of significant and accessible N—H bonds. These deficiencies make the catalysts inefficient to be used in the Knoevenagel condensation.

### SUMMARY

A discovery has been made that addresses the problems associated with carbon nitride (CN) catalysts used for the Knoevenagel condensation. The discovery is premised on the preparation of a mesoporous material that includes a three dimensional (3D) cyanamide based mesoporous graphitic carbon nitride (MGCN) matrix having a range of unique and beneficial properties that are tunable according to the reactions conditions employed. These properties include a d spacing of 89 to 92, a surface area of 195 to 300 m²/g, a pore volume of 0.40 to 0.80 cm³ g⁻¹, a tunable pore size, or any combination thereof. Further characterization of the mesoporous material shows a highly basic, well ordered, 3D-cubic Ia3d symmetric mesoporous CN with graphitic pore walls, very high nitrogen content (e.g., a carbon to nitrogen (C:N) atomic ratio of at least 0.7 or N:C atomic ratio of 1.42), and sheets of three dimensionally arranged s-heptazine units. Without wishing to be bound by theory, the combination of these properties along with facile preparation from inexpensive and nontoxic precursors can make the current MGCN material suitable for a catalytic aldol reaction. Notably, the MGCN material of the current invention has increased numbers and accessibility of N—H functionality that provides an excellent room temperature catalyst for the Knoevenagel condensation affording reaction products in greater than 92% yield with at least 98% selectivity. The MCN catalyst of the present invention can also be used as catalyst for various other basic catalysed reactions such as aldol condensation of aromatic and aliphatic aldehydes with ketones, nitro-aldol condensation of aromatic and aliphatic aldehydes with nitromethane, transesterification of β-ketoesters, Suzuki coupling reaction, oxidative dehydrogenation of alkanes, aza-Michael addition reaction of amines, etc. The functionalized MCN can also be used as catalysts for the conversion of benzaldehyde dimethylacetal to benzylidine malononitride.

In a particular embodiment of the current invention, there is described a mesoporous carbon nitride (CN) material. The mesoporous CN material can include a mesoporous graphitic carbon nitride material (MGCN) that includes a three dimensional cyanamide based mesoporous graphitic carbon nitride material (MGCN). The MGCN can include sheets of three dimensionally arranged s-heptazine units. In addition, the MGCN material can have a pore volume between 0.40 and 0.80 cm³ g⁻¹ and a surface area of 195 to 300 m² g⁻¹. In one aspect, the mesoporous CN material can have a d spacing of 89 to 92. In certain aspects, the mesoporous CN material can have a pore volume between 0.70 and 0.80 cm³ g⁻¹ and a surface area of 275 to 300 m² g⁻¹. In other aspects, the mesoporous CN material of the current invention can be used as a catalyst in aldol chemistry, such as the Knoevenagel reaction.

According to another particular embodiment of the current invention, a condensation reaction process is described. The process can include (a) contacting the mesoporous graphitic carbon nitride material with a carbonyl containing compound and an activated methylene containing compound forming a reactant mixture; and (b) subjecting the reactant mixture to conditions in which the carbonyl and methylene group are condensed forming a carbon-carbon bond. In some instances, the process can be performed at a temperature of 10 to 30° C. resulting in a yield of at least 92% and a selectivity of at least 98%. In a specific embodiment, the condensation process is an aldol condensation, such as the Knoevenagel reaction.

In other embodiments, a method of producing a mesoporous graphitic carbon nitride material of the present invention is described. The method can include (a) mixing a calcined KIT-6 template with an aqueous cyanamide solution forming a template reactant mixture; (b) subjecting the template reactant mixture to conditions to form a templated carbon nitride composite; (c) heat treating the template carbon nitride composite to a temperature of 450 to 550° C. to form a mesoporous graphitic carbon nitride material/KIT-6 (MGCN-KIT-6) complex; and (d) removing the KIT-6 template from the mesoporous graphitic carbon nitride material/KIT-6 complex to produce a three dimensional cyanamide based mesoporous graphitic carbon nitride material that includes sheets of three dimensionally arranged s-heptazine units. In one aspect of the method, the step (b) conditions can include holding the solution at room temperature (e.g., 20 to 30° C.), collecting the templated carbon nitride composite by centrifugation to collect a precipitate, and then drying the precipitate under vacuum. In another aspect, the aqueous cyanamide solution can be 40-60% cyanamide by weight, preferably about 50% cyanamide by weight. In some aspects, the heat treating step (c) is performed under a nitrogen flow at about 500° C. The nitrogen flow can be at 50 mL per minute, and the temperature of heating step (c) can be achieved using a heating rate of about 2.0° C. per minute. In another embodiment, a method to produce the KIT-6 template is described. The method can include (a) reacting a polymerization solution including amphiphilic triblock copolymer and tetraethyl orthosilicate (TEOS) at a predetermined reaction temperature to form a KIT-6 template; (b) drying the KIT-6 template at 90° C. to 110° C.; and (c) calcining the dried KIT-6 template in air at 500 to 600° C., preferably 540° C. to form a calcined KIT-6 template. The predetermined reaction temperature of step (a) can determine the pore size of the KIT-6 template. In one aspect, the method further includes heating the dried KIT-6 template at a synthesis temperature of about 100 to 200° C. prior to calcining step (c). In other aspects, the method includes incubating the dried KIT-6 template at a synthesis temperature of about 150° C. prior to calcining step (d).

Other embodiments of the invention are disclosed throughout this application. Any embodiment disclosed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions of the invention can be used to achieve methods of the invention.

The following includes definitions of various terms and phrases used throughout this specification.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The MGCN materials of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the catalysts of the present invention are their abilities to catalyze aldol condensations.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein. The drawings may not be to scale.

FIG. 1 is a schematic representation for the preparation of cyanamide based 3D cubic mesoporous graphitic carbon nitride (MGCN) of the present invention using KIT-6.

FIG. 2 depicts a system for CO₂ capture and activation to form reaction products.

FIG. 3 shows 3(A) low angle powder X-ray diffraction (XRD) patterns of KIT6 materials synthesized at different temperature KIT6-100, KIT6-130 and KIT6-150; 3(B) low angle powder XRD patterns of KIT6-150 and MGCN-6-150.

FIG. 4 shows 4(A) low angle powder XRD patterns of mesoporous graphitic carbon nitride (MGCN) with various pore diameters prepared from KIT-6-X templates: MGCN-6-100, MGCN-6-130 and MGCN-6-150, 4(B) wide angle XRD patterns of mesoporous graphitic carbon nitride (MGCN) with various pore diameters prepared from KIT-6-X templates: MGCN-6-100, MGCN-6-130 and MGCN-6-150.

FIG. 5 shows 5(A) nitrogen adsorption-desorption isotherms of mesoporous graphitic carbon nitride with various pore diameters (open symbols: adsorption, closed symbols: desorption; circles: MGCN-6-100, triangles: MGCN-6-130, and squares: MGCN-6-150), 5(B) adsorption pore-size distributions of mesoporous graphitic carbon nitride with various pore diameters (triangles: MGCN-6-130 and squares: MGCN-6-150) using from Barrett-Joyner-Halenda (BJH) analysis.

FIG. 6 shows 6(A) nitrogen adsorption-desorption isotherms and 6(B) adsorption pore size distributions of KIT6-150 and MGCN-6-150 (closed symbols: desorption; open symbols: adsorption; circles: KIT6-150 and squares: MGCN-6-150) using BJH analysis.

FIG. 7 shows high resolution tunneling electron microscopy (HRTEM) images of MGCN-6-150 at various magnifications 7(A) scale bar 100 nm and 7(B) scale bar 50 nm.

FIG. 8 shows high resolution scanning electron microscopy (HRSEM) images of MGCN-6-150 at 8(A) 50× and 8(B) 100×.

FIG. 9 shows energy dispersive spectrometry (EDAX) of MGCN-6-150.

FIG. 10 shows (10A and 10B) elemental mapping of MGCN-6-150.

FIG. 11 shows the Fourier transform infrared (FT-IR) spectrum of MGCN-6-150.

FIG. 12 shows ultra violet visible diffuse reflectance spectroscopy (UV-Vis DRS) patterns of mesoporous graphitic carbon nitride (MGCN) with various pore diameters prepared from KIT-6-X (X=100, 130, 150° C.) templates: MGCN-6-100, MGCN-6-130 and MGCN-6-150.

FIG. 13 shows the C1s and N1s X-ray photoelectron spectroscopy (XPS) spectra of MGCN-6-150.

FIG. 14 shows the XPS survey spectrum of mesoporous graphitic carbon nitride (MGCN-6-150).

FIG. 15 shows graphical progress of a Knoevenagel condensation reaction using MGCN-6 materials.

### DETAILED DESCRIPTION

A discovery has been made that provides a mesoporous graphitic carbon nitride (MGCN) material having the appropriate characteristics for use as a catalyst in synthetic chemistry (e.g., an aldol reaction, or modified aldol condensations, the Knoevenagel condensation, etc.). The discovery is premised on a preparation method that provides a three dimensional cyanamide based mesoporous graphitic carbon nitride matrix that offers increased accessibility and numbers of pores containing reactive amine functionality useful for catalysis. In certain aspects, the tuning of the MGCN material can be accomplished by controlling the pore size and other dimensions of the mesoporous MGCN material.

These and other non-limiting aspects of the present invention are discussed in further detail in the following sections with reference to the Figures.

### A. Mesoporous Graphitic Carbon Nitride Materials

Certain embodiments are directed to a mesoporous material based on cyanamide (NCNH₂). Such a material can have a highly ordered three dimensional mesoporous graphitic carbon nitride (MGCN) based hybrid material having crystalline wall structure with very high nitrogen content, high surface area, and large pore volume. In particular aspects, the MGCN material can have a body centered cubic Ia3d structure with tunable pore diameters prepared by a hard template approach from three dimensional mesoporous silica, (e.g., KIT-6) through a temperature-induced polycondensation of NCNH₂. The MGCN material prepared in this way, and where the template is later removed, is referred to as MGCN. For simple cubic structures, d spacing is a measure of the distance between adjacent repeating planes. The structure of the resulting MGCN material contains sheets of three-dimensionally arranged s-heptazine units that can be held together by covalent bonds between C and N atoms. A heptazine, or tri-s-triazine or cyamelurine, is a type of chemical compound that has planar triangular core group, C₆N₇, or three fused triazine rings, with three substituents at the corners of the triangle. When heptazine is polymerized with the tri-s-triazine units linked through an amine (NH) link can be referred to as “melon”. In a non-limited embodiment, a representative trimeric heptazine denoted s-heptazine having general molecular formula C₃N₄ containing increased numbers and accessible N—H and NH₂ functionality can have the following structure:

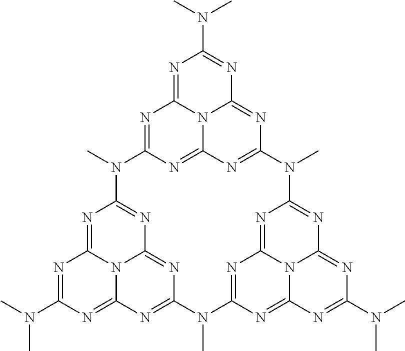

In such a configuration, the d spacing of the MGCN-6 material can range from 80 to 100 or any value or range there between (e.g., 81, 82, 83, 84, 85, 86, 87, 88, 89, 90, 91, 92, 93, 94, 95, 96, 97, 98, or 99). Preferably, the d spacing is 89 to 92. In another aspect, the MGCN-6 material can have a pore size or pore diameter of 1 nm, 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, or 30 nm. Specifically, the pore size can be varied based on the desired function of the MGCN material. For example, during use as a catalyst in the Knoevenagel condensation, the pore size can be selected based on the molecular size of the starting materials employed or products formed. In this way, the MGCN materials of the current invention can be optimized for particular substrates and/or products to produce maximum reaction efficiency (e.g., yields, selectivity, TON, etc.). Pore size tuning can also permit molecular recognition properties based on molecular size of substrate, transition state complex, or products, such that the current MGCN materials can be employed as a substrate selective catalyst. In certain aspects, the pore volume of the MGCNmaterial can range from 0.40 to 0.80 cm³ g⁻¹ or any value or range there between (e.g., 0.40, 0.41, 0.42, 0.43, 0.5, 0.51, 0.52, 0.53, 0.54, 0.55, 0.56, 0.57, 0.58, 0.59, 0.60, 0.61, 0.62, 0.63, 0.64, 0.65, 0.67, 0.68, 0.69, 0.70, 0.71, 0.72, 0.73, 0.74, 0.75, 0.76, 0.77, 0.78, 0.79, or 0.80 cm³ g⁻¹). Preferably, the pore volume is 0.40 to 0.80 cm³ g⁻¹ or 0.70 to 0.80 cm³ g⁻¹. The surface area of the MGCN-6 materials can be from 195 to 300 m² g⁻¹ or any range or value there between (e.g., 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 243, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 m² g⁻¹). Preferably, the surface area ranges from 195 to 300 m² g⁻¹ or from 275 to 300 m² g⁻¹. Without being limited by theory, the MGCN material of the present invention has highly basic characteristics that provide its unique and beneficial properties. The highly basicity can be attributed to the increased presence of primary and secondary amines (i.e., NH and NH₂) functionality on the surface and/or within the pores of the MGCN-6 material. The carbon to nitrogen atomic ratio (C:N) can be 0.7 to 0.8, 0.72 to 0.75, or 0.71, 0.72, 0.73, 0.74, 0.75, 0.76, 0.78, 0.79, 0.80. The nitrogen to carbon atomic ratio can be 1.25 to 1.42, 1.33 to 1.39, or greater than, equal to, or between any two of 1.25, 1.30, 1.35, 1.4, and 1.42.

### B. Method of Making

The MGCN material can be formed by nanocasting using a template. Nanocasting is a technique to form periodic mesoporous framework using a hard template to produce a negative replica of the hard template structure. A molecular precursor can be infiltrated into the pores of the hard template and subsequently polymerized within the pores of the hard template at elevated temperatures. Then the hard template can be removed by a suitable method. This nanocasting route is advantageous because no cooperative assembly processes between the template and the precursors are required. A hard template can be a mesoporous silica. In one aspect, the mesoporous silica can be KIT-6, MCM-41, SBA-15, TUD-1, HMM-33, etc., or derivatives thereof prepared in similar manners from tetraethyl orthosilicate (TEOS) or (3-mercaptopropyl) trimethoxysilane (MPTMS). In certain aspect, the mesoporous silica is a 3D-cubic Ia3d symmetric silica, such as KIT-6 which contains interpenetrating cylindrical pore systems. Highly ordered mesoporous silica can be obtained under various conditions using inexpensive materials.

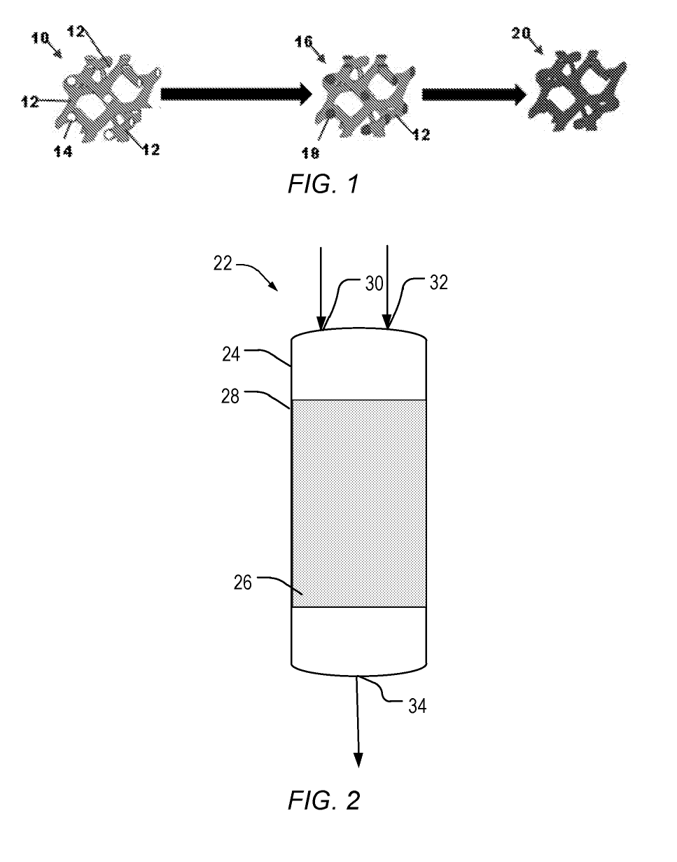

FIG. 1 is a schematic representation of one embodiment of a method for producing a MGCN material by using a hard templating approach, also called a replica approach, as described herein. Template 10 (e.g., calcined KIT-6) can include canal 12 and pores 14. Canal 12 is representative of the pore volume of template 10. Pores 14 can be filled corresponding carbon nitride precursor material 16 to form a template/carbon nitride precursor material. By way of example, an aqueous solution of cyanamide can be added to a KIT-6. The template/carbon nitride precursor material can undergo a thermal treatment to polymerize the precursor inside the pore of the material to form template/CN composite 16 having canal 12 and polymerized CN material 18. Template/CN composite 16 can be subjected to conditions sufficient to remove the template 10 (e.g., KIT-6), and form the MGCN-6 material 20 of the present invention. By way of example, the template 10 can be dissolved using an HF treatment, a very high alkaline solution, or any other dissolution agent capable of removing the template and not dissolving the CN framework. The kind of template and the CN precursor used influence the characteristics of the final material. By way of example, various KIT-6 with various pore diameters can be used as templates. In certain aspects, the pore size of the KIT-6 template can be tuned and cyanamide can be used to produce a high nitrogen content.

In one non-limiting embodiment, step one of a method to prepare a MGCN-6 material can include obtaining an template reactant mixture including a calcined mesoporous KIT-6 template having a selected porosity and an aqueous cyanamide solution. Preferably, the wt. % ratio of cyanamide and KIT-6 template in the reactant mixture is 10:1. In some instances, obtaining the template reactant mixture includes adding calcined KIT-6 to a cyanamide in aqueous solution. The aqueous cyanamide solution can be 40-60% cyanamide by weight or about 50% cyanamide by weight. In other instances, the template reactant mixture can be suspension or a gel. In step 2 of the method, the template reactant mixture can be contact at room temperature (e.g., 20° C. to 30° C., or 22 to 28° C., or about 25° C.) to form a templated carbon nitride (CN) composite. The time of contact can be 0.25, 0.50, 0.75, 1, 2, 3, 4, 5, 6, 7, 8, 9, 10, 11, or 12 hours. Typically the time of contact is about 1 hour or until complete penetration of the cyanamide into the template has occurred. Step 3 of the method can include collecting the template CN composite. Collection can include centrifugation to produce a precipitate that can be collected by filtration and placed under vacuum (e.g., in a vacuum desiccator) for an appropriate amount of time (e.g., 24 hours) to obtain a dry material. Centrifugation of the template CN composite can occur for 10, 20, 30, 40, 50, or 60 minutes at 5,000, 6,000, 7,000, 8,000, 9,000, 10,000, 11,000, 12,000, 13,000, 14,000, or 15,000 rpm, including all values and ranges there between. Preferably, centrifugation can be carried out for 30 minutes at 10,000 rpm. Step 4 of the method can include polymerization of the templated CN composite. The templated CN composite can be heated under a flow of inert atmosphere (e.g., argon, nitrogen, or mixtures thereof) to a temperature of 450 to 550° C., preferably about 500° C., for a period of time (e.g., 4 hours) to form a mesoporous graphitic carbon nitride material/KIT-6 complex (MGCN-KIT-6). In some aspects, the template CN composite can be heated under inert gas flow to temperature at a rate of about 1, 2, 3, 4, 5, or 6° C. per minute, preferably 2° C. per minute. The inert gas flow can be at about 10, 20, 30, 40, 50, 60, 70, 80, 90 or 100 mL per minute, including all values and ranges there between. Specifically, the inert gas is nitrogen and the nitrogen flow is 50 mL per minute. In step 5 of the method, the KIT-6 can be removed by dissolving the KIT-6 template from the MGCN-KIT-6 complex to form the three dimensional cyanamide based MGCN-6 material of the present invention containing sheets of three dimensionally arranged s-heptazine units. In some aspects, hydrofluoric acid or other suitable solvent or treatment can be used that dissolves the KIT-6 without dissolving the CN framework. The method can further include collecting the MGCN-6 based hybrid material by filtration, washing with ethanol, and drying at 100° C. In a further aspect, the filtered, washed, and dry material can be ground to a powder and/or purified and/or stored and/or used directly in subsequent applications (e.g., Knoevenagel condensation).

A KIT-6 template can be produced by first obtaining a polymerization solution including an amphiphilic triblock copolymer dispersed in an aqueous hydrogen chloride solution with 1-butanol and tetraethyl orthosilicate (TEOS) to form a polymerization mixture. In a second step the polymerization mixture can be reacted by heating at a predetermined synthesis temperature to form a KIT-6 template, wherein the predetermined temperature determines the pore size of the KIT-6 template. The polymerization mixture can be heated at a synthesis temperature of about 100 to 200° C., or any value or range there between (e.g., 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 143, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, or 199° C.). For the general formula KIT-6-X, X represents the incubation temperature. For example, in certain aspects the polymerization mixture can be heated at a synthesis temperature of about 100, 130, or 150° C. to yield corresponding KIT-6 templates denoted KIT-6-100, KIT-6-130, and KIT-6-150, respectively. Preferably, the incubation temperature is 100° C. The formed KIT-6 template can then be dried at 90° C. to 110° C., preferably 100° C. In a final step, the dried KIT-6 template can be calcined. Calcination includes heating the KIT-6 template to about 500 to 600° C. or any value or range there between (e.g., 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 543, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, or 600° C., preferably 540° C.) in air to decompose the triblock copolymer

A non-limiting example of producing a KIT-6 template includes mixing Pluronic P-123 in aqueous HCl with stirring at 35° C. until dissolution. n-Butanol (1-butanol) can then be added with continued stirring and after 1 hour tetraethyl orthosilicate (TEOS) can be added and the resulting mixture can be vigorously stirred at 35° C. for 24 hours. The mixture can then be aged at 150° C. for 24 h under static conditions and resulting colorless solid, and then be filtered at temperatures of 50° C. without washing under, and dried in oven at 100° C. for 24 h and then calcined in air at 540° C.

### C. Use of the Mesoporous Graphitic Carbon Nitride Materials

The three dimensional cyanamide based mesoporous graphitic carbon nitride matrix material can be used in many applications, such as capture and activation CO₂, absorption of bulky molecules, catalysis, light emitting devices, as a storage material, sensing device, etc. Specifically, the mesoporous material of the current invention can be used as a catalyst in the Knoevenagel condensation. An example of the Knoevenagel reaction is shown in the scheme below. In the scheme, the catalyst of the present invention is represented by the (—NH—) compound, which can be a primary or secondary amine. B3 refers to a base, IHB refers to a protonated base, and Et refers to an ethyl group.

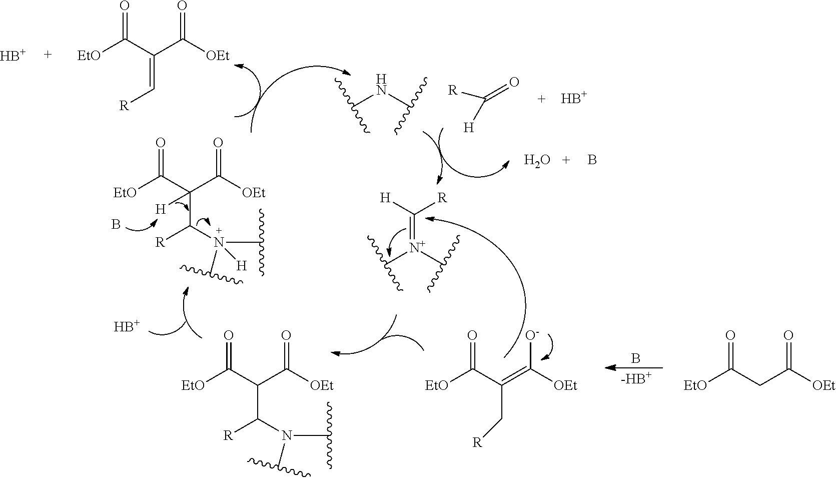

According to one embodiment of the present invention, a process for a condensation reaction (e.g., Knoevenagel reaction) is described. Other reactions (e.g. Hantzsch pyridine synthesis, Gewald reaction, Feist-Benary furan synthesis, Doebner modification, Weiss-Cook reaction, and other various aldol-type condensations) that contain a Knoevenagel reaction mechanism or condensation-like mechanism are also contemplated by the current embodiments. In step one of the process, a MGCN material (e.g., MGCN-6 material) is contacted with a carbonyl containing compound and an activated methylene containing compound forming a reactant mixture. The carbonyl compound can be a aldehyde or a ketone and the activated methylene can be have the general formula Z—CH₂—Z or Z—CHR—Z (e.g., malononitrile, diethyl malonate, Meldrum's acid, ethyl acetoacetate, malonic acid, or cyanoacetic acid, etc.), or have the general formula Z—CHR₁R₂ (e.g., nitromethane, nitroethane, nitropropane, etc.), where Z is an electron withdrawing group. The electron withdrawing group is sufficient to facilitate deprotonation to the enolate ion even with a mild base, such that self-condensation of the aldehyde or ketone does not occur. In a step 2 of the condensation process, the reactant mixture can be held under conditions in which the carbonyl and methylene group are condensed forming a carbon-carbon bond. The incubation conditions can include a temperature and time. The temperature range for the incubation can be from 10° C. to 30° C. and all ranges and temperatures there between (e.g., 10, 11, 12, 13, 14, 15, 16, 17, 18, 19, 20, 21, 22, 23, 24, 25, 26, 27, 28, 29, or 30° C.). The time of incubation can be from 10 minutes to 300 minutes and all ranges and temperatures there between (e.g., 10, 20, 30, 40, 50, 60, 70, 80, 90, 100, 110, 120, 130, 140, 150, 160, 170, 180, 190, 200, 210, 220, 230, 240, 250, 260, 270, 280, 290, or 300 minutes). The conditions for the condensation reaction process can be varied based on the source and composition of feedstock and/or the type of the reactor used. An advantage of the using the MGCN materials in the condensation reaction process of the current invention includes obtaining high yields (e.g., at least 90, 91, 92, 93, 94, 95, 96, 97, 98, or 99% yield) and product selectivities (e.g., at least 95, 96, 97, 98, 99, or 100% selectivity). In a preferred embodiment, the condensation process is a Knoevenagel condensation.

According the other embodiment, a system for a Knoevenagel condensation to form a reaction product is described. Referring to FIG. 2, system 22 is system used to contact a MGCN material (e.g., MGCN-6 material) with a carbonyl containing compound and an activated methylene containing compound to catalyze the forming of a condensation mixture. Reactor 24 can include MGCN material 26 in reaction zone 28. A carbonyl containing compound (e.g., a aldehyde or ketone) can enter reactor 24 via inlet 30 and an activated methylene containing compound (e.g., malononitrile, diethyl malonate, Meldrum's acid, ethyl acetoacetate, malonic acid, cyanoacetic acid, or a nitroalkane, etc.) can enter reactor 24 via inlet 32. The carbonyl and activated methylene containing compounds can mix in reactor 24 to form a reactant mixture. In some embodiments, the carbonyl and activated methylene containing compounds can be provided as one stream to reactor 24. In reaction zone 28 as the carbonyl and activated methylene containing compounds pass over the MGCN material (e.g., MGCN-6 material), the basic nitrogen sites on the MGCN material can activate the carbonyl compound to react with the activated methylene compound to form a condensation product. By way of example, benzaldehyde and malononitrile in ethanol at room temperature can be contacted with the MGNC material to produce 2-benzylidenemalononitrile. The reactor 24 can be heated or cooled under desired pressures and temperatures to further promote the condensation reaction. The reaction product can exit reactor 24 via product outlet 34 and be collected, stored, transported, or provided to other units for further processing. If necessary, the reaction product can be purified. For example, unreacted carbonyl and activated methylene containing compounds can be separated (e.g., sent to a separation system) and recycled to reactor 24. System 22 can also include a heating source (not shown). The heating source can heaters, heat exchange systems or the like, and be configured to heat the reaction zone 42 or the separation zone to a temperature sufficient to perform the desired reaction or separation.

### EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Materials.

Tetraethyl orthosilicate (TEOS), cyanamide 50 wt. % in water solution, 1-butanol, and triblock copolymer poly(ethylene glycol)-block-poly(propylene glycol)-block-poly(ethylene glycol) (Pluronic P-123, molecular weight 5800 g mol⁻¹, EO₂₀PO₇₀EO₂₀), which were obtained from Sigma-Aldrich® (U.S.A). Ethanol and hydrofluoric acid (HF) were purchased from Wako Pure Chemical Industries (U.S.A.). All the chemicals were used without further purification. Doubly deionized water has been used throughout the synthesis process.

### Example 1

(Preparation of Mesoporous 3D KIT-6 Silica Template with Different Pore Diameters)

KIT-6 having different pore diameters was synthesized by using a P123 and n-butanol mixture as the structure directing agent at different synthesis temperatures. In a typical synthesis, P123 (4.0 g) was dispersed in a water (144 g) and HCl solution (7.9 g), and stirred for 3 hours at 35° C. to obtain an aqueous P-123 homogeneous solution. 1-Butanol (4.0 g) was added to the aqueous P-123 homogeneous solution and the mixture was stirred for a further 1 hour. TEOS (8.6 g) is then added and stirring was continued at 35° C. for 24 hours to produce a reaction mixture. Subsequently, the reaction mixture was aged at 100° C. for 24 h under static conditions. At these conditions a white solid product was formed. The white solid product was filtered at a 50° C. or less without washing, and then dried at 100° C. for 24 hours in an air oven. Finally, the product was calcined at 540° C. in air to decompose the triblock copolymer. KIT-6 silica template materials with different pore diameters were synthesized at the synthesis temperatures of 100, 130, and 150° C. The samples were labeled KIT-6-X, where X denotes the synthesis temperature of 100, 130, and 150° C.

### Example 2

### Synthesis of MGCN-6 Materials

Highly ordered mesoporous graphitic carbon nitride materials having very high nitrogen content were prepared using mesoporous silica with various pore diameters as templates. The calcined KIT-6-100, -130, or -150 (1.0 g) of Example 1 was added to cyanamide (10.0 g, 50 wt. % in water solution). The resultant mixture was stirred at room temperature for 1 hour or until complete penetration has occurred. Then, the resulting mixture was centrifuged for 30 min (10000 rpm). The resultant precipitate was dried in vacuum desiccator for 24 hours. The template-carbon nitride polymer composites were then heat-treated in a nitrogen flow of 50 mL per minute at 500° C. with a heating rate of 2.0° C. min⁻¹ and kept under these conditions for 4 hours to carbonize the polymer. The high nitrogen content mesoporous graphitic carbon nitride (MGCN-6) based hybrid material was recovered by filtration after dissolution of the KIT-6 silica framework in hydrofluoric acid (5 wt. %), washed several times with ethanol, and dried at 100° C.

### Example 3

### Characterization of MGCN-6 Materials

Mesoporous KIT-6 silica materials with different pore diameters were used as the hard template in the preparation of mesoporous graphitic carbon nitride (MGCN-6). The material was further characterized as follows.

1. X-Ray Diffraction Analysis

The ordered mesoporous structure of MGCN-6 materials along with the parent silica templates was investigated by powder XRD analysis. Powder XRD patterns were recorded on Rigaku Ultima+diffractometer using CuKα (λ=1.5408 Å) radiation. Low angle powder x-ray diffractograms were recorded in the 20 range of 0.6-6° with a 2θ step size of 0.0017 and a step time of 1 sec. In the case of wide angle X-ray diffraction, the patterns were obtained in the 20 range of 10-80° with a step size of 0.0083 and a step time of 1 sec.

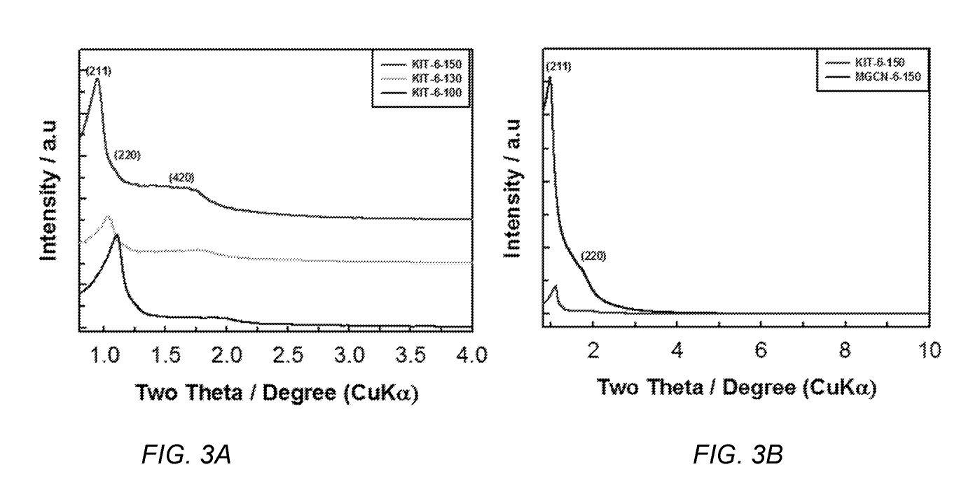

FIG. 3A shows the low angle powder XRD diffraction patterns for a series of the KIT-6 materials synthesized at synthesis temperatures of 100° C. (bottom), 130° C. (middle) and 150° C. (top) for a period of 24 hours. All of the samples exhibited a sharp well-ordered peak indexed at 211 and several higher order peaks below 4°, indicating an excellent structural ordering with a body centered cubic Ia3d space group.

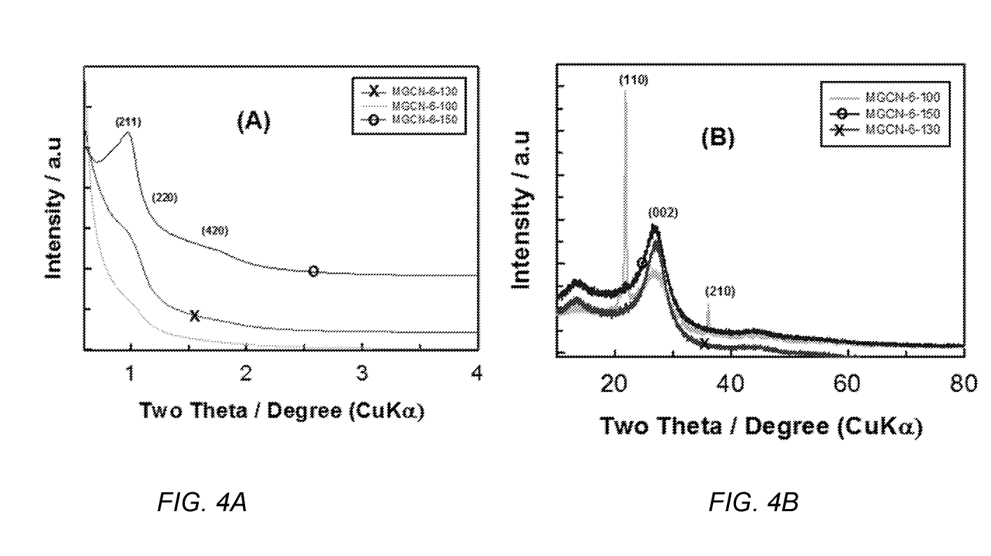

The XRD peaks shift gradually towards lower 20 values on increasing the temperature of the hydrothermal treatment from 100 to 150° C., which reflected an increase in the d-spacing. Notably, the pore diameter of the KIT-6 materials increased significantly with increasing the hydrothermal synthesis temperature (Table 1), which is consistent with the lattice expansion observed from the XRD patterns in FIG. 3A. FIG. 3B shows the lower angle powder XRD patterns of MGCN-6-150 and KIT-6-150. FIG. 4A shows the low angle powder XRD patterns of MGCN-6 made from the corresponding KIT-6 templates of FIG. 3A. FIG. 4B shows wide angle powder XRD patterns of materials made from the corresponding KIT-6 templates of FIG. 3A. As shown in FIG. 3B, only MGCN-6-150 exhibited a sharp peak that can be indexed at 211 plane of the highly ordered three-dimensional cubical meso-structure with the space group of Ia3d, similar to the XRD pattern of the parent KIT-6 mesoporous silica template which consists of a cubical arrangement of pores. Both MGCN-6-100 and MGCN-6-130 show only a broad peak that was indexed as 211 diffractions. This may be attributed to the high nitrogen content which needs a comparatively larger pore diameter for an ordered structure which ordering of pores was found to increase from MGCN-6-100 to MGCN-6-150 with increasing the pore diameter of the mesoporous silica hard template KIT-6. Notably, an increase in the pore diameter of the templates causes a shift of the peak towards lower 20 values, which provides evidence of an increase in d-spacing (Table 1).

The wide-angle XRD pattern (FIG. 4B) of MGCN-6-130 and MGCN-6-150 exhibits the typical graphitic 002 basal plane diffraction peak at a 20 value of 27.14° (d=0.327 nm) which reveals the presence of a turbostatic ordering of the carbon and nitrogen atoms resulting in a highly crystalline wall in the structure. Another pronounced peak was found at 13.40 indexed at the 100 plane, which corresponded to an in-plane structural packing motif. MGCN-6-100 showed diffractions of 110 and 210 planes along with graphitic 002 basal plane resulting from the highly crystalline nature of the material.

TABLE 1

| | | | | Specific | | |
|---|---|---|---|---|---|---|
| | | | | surface | Pore | Pore |
| Sample | Sample | 2 theta | d | area | volume | diameter |
| No. | Name | (Degree) | Spacing | (m² gm⁻¹) | (cc gm⁻¹) | (nm) |
| 1 | KIT-6-100 | 1.43 | 80.33 | 721.5 | 0.99 | 7.2 |
| 2 | KIT-6-130 | 1.03 | 85.07 | 752.3 | 1.23 | 9.6 |
| 3 | KIT-6-150 | 0.64 | 93.93 | 781.6 | 1.5 | 11.02 |
| 4 | MGCN-6-100 | 0.985 | 89.64 | 196.1 | 0.45 | 2.9 |
| 5 | MGCN-6-130 | 0.980 | 90.08 | 258.3 | 0.72 | 3.5 |
| 6 | MGCN-6-150 | 0.965 | 91.49 | 280.5 | 0.769 | 4.2 |

Total pore volumes were estimated from the adsorbed amount at a relative pressure of P/P⁰ = 0.99.
Pore diameters derived from the adsorption branches of the isotherms by using the BJH method.

2. Nitrogen Adsorption-Desorption and BJH Adsorption.

The textural parameters and the mesoscale ordering of the MGCN materials prepared using KIT-6 materials with different pore diameters as templates were analyzed by nitrogen adsorption-desorption analysis. Nitrogen adsorption-desorption isotherms were measured by using Quantachrome sorption analyzer at −196° C. All samples were out-gassed for 12 hours at high temperatures under vacuum (p<1×10⁵ h·Pa) in the degas port of the adsorption analyzer. The specific surface area was calculated using the Brunauer-Emmett-Teller (BET) method. The pore size distributions were obtained from either adsorption or desorption branches of the isotherms using Barrett-Joyner-Halenda (BJH) method.

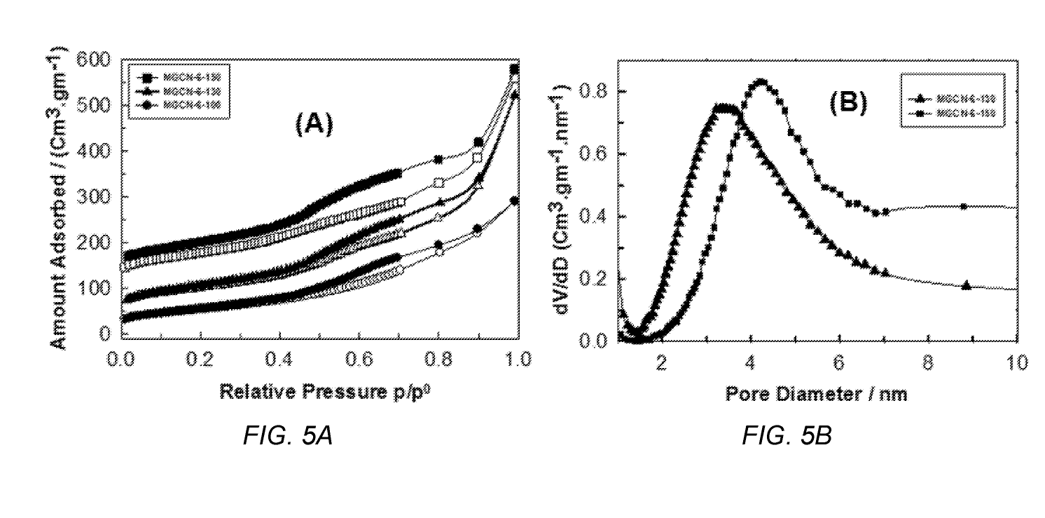

FIG. 5A shows the nitrogen adsorption isotherms of the MGCN-6-100, MGCN-6-130 and MCN-6-150 samples. All of the isotherms were of type IV according to the IUPAC classification and featured capillary condensation in the mesopores, which indicates the presence of a well-ordered array of mesopores in all of the samples. The textural parameters such as the specific surface area, specific pore volume and the pore diameter of the MGCN-6 samples are also given in Table 1. The change in the pore diameter of the MGCN-6 materials upon increasing the pore diameter of the template was evident from the pore-size distribution curve. FIG. 5B shows the pore-size distribution of MGCN-6-130 and MGCN-6-150 from the adsorption branch. All of the samples show a main peak that originates from the mesopores formed after dissolution of the silica matrix from the template. The pore diameter of the MGCN-6 materials increases with increasing the pore diameter of the silica templates used.

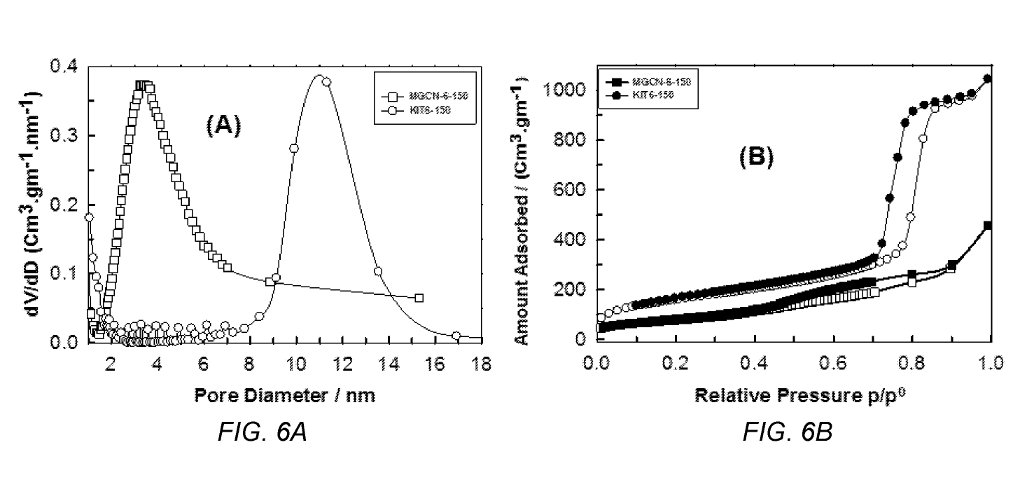

Also noteworthy, the BJH adsorption pore-size distribution of MGCN-6-150 was much larger than that of MGCN-6-130 and MGCN-6-100. Among the MGCN-6 samples prepared using KIT-6-X as the template, MGCN-6-150 exhibits a very-large pore diameter, which was around 4.0 nm. This could be a result of the incomplete filling of the CN polymer matrix in the ultra-large mesopores of KIT-6-150 as the same quantity of cyanamide precursor was used for filling the mesopores of the all the templates with different pore diameters. Without wishing to be bound by theory, it is believed that MGCN-6-100 does not exhibit a uniform pore size distribution due to its highly crystalline nature and disordered pores arrangement. Notably, the specific surface area and the pore volume of MGCN-6-150 were higher as compared to those for the MGCN-6-100 and the MGCN-6-130. The specific surface area and the specific pore volume of MGCN-6-150 were 280 m² g⁻¹ and 0.78 cm³ g⁻¹ respectively, whereas MGCN-6-100 and MCN-6-130 possessed specific surface areas of 196 and 258 m² g⁻¹ respectively, and specific pore volumes of 0.46 and 0.74 cm³ g⁻¹, respectively. The specific surface and pore volume of MGCN-6 materials were small when compared with parent silica template. Without wishing to be bound by theory, it is believed that the polycondensation of cyanamide to polymeric melon nano-composites with a large number of aromatic rings does not create much microporosity since it is very difficult to break the aromatic ring at the polymerization temperature of 550° C. in an inert atmosphere, leading to a low specific surface area and pore volume in the material. The nitrogen adsorption-desorption isotherms and BJH adsorption pore size distributions of MGCN-6-150 and KIT-6-150 are shown in FIGS. 6A and 6B. The pore size distribution of KIT-6-150 appears narrow and the peak centered at about 8.0 nm whereas MGCN-6-150 exhibits a narrow peak centered around 4.0 nm, which was much larger than the wall thickness of the KIT-6 mesoporous silica (about 3.2 nm).

3. HRTEM and HRSEM

HRTEM images were obtained using a high-resolution transmission electron microscope JEOL-3100FEF, equipped with a Gatan-766 electron energy-loss spectrometer (EELS). The preparation of the samples for 1-IRTEM analysis involved sonication in ethanol for 5 min and deposition on a copper grid. The accelerating voltage of the electron beam was 200 kV.

HRTEM images were obtained using a JEOL-3000F and a JEOL-3100FEF field emission high-resolution transmission electron microscope equipped with a Gatan-766 electron energy-loss spectrometer with an electron beam accelerating voltage of 200 kV.

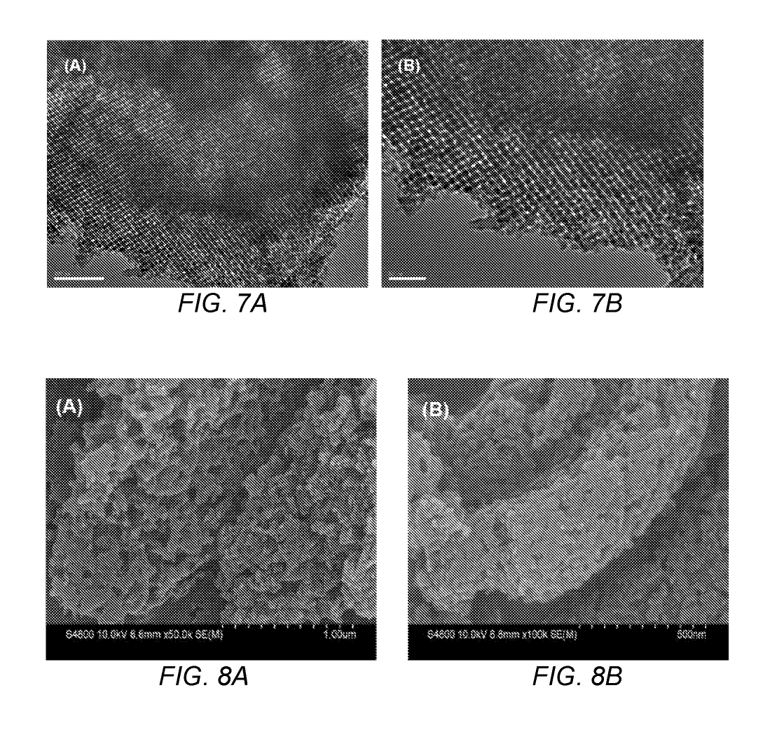

FIGS. 7A and 7B shows the HRTEM image of MGCN-6 taken along [220], in which the bright contrast strips on the image represent the crystalline pore wall images and the dark contrast cores display empty channels, shows a well-ordered mesoporous structure with a regular intervals of linear array of mesopores throughout the samples, which is characteristic of well-ordered KIT-6 mesoporous silica. This demonstrates that 3D mesoporous graphitic carbon nitride with body centered cubic Ia3d structure type has been replicated from the KIT-6 mesoporous silica template with cyanamide precursor.

4. Elemental Analysis and EDAX

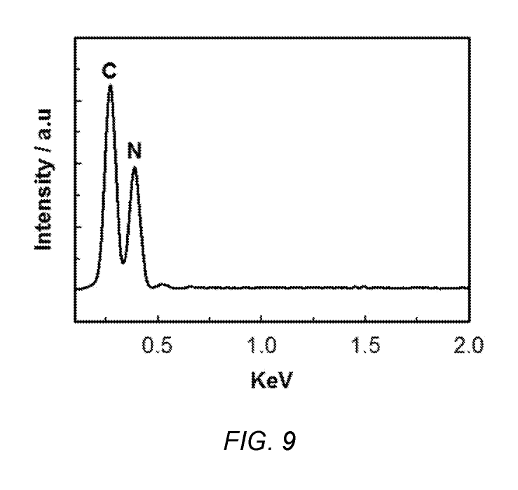

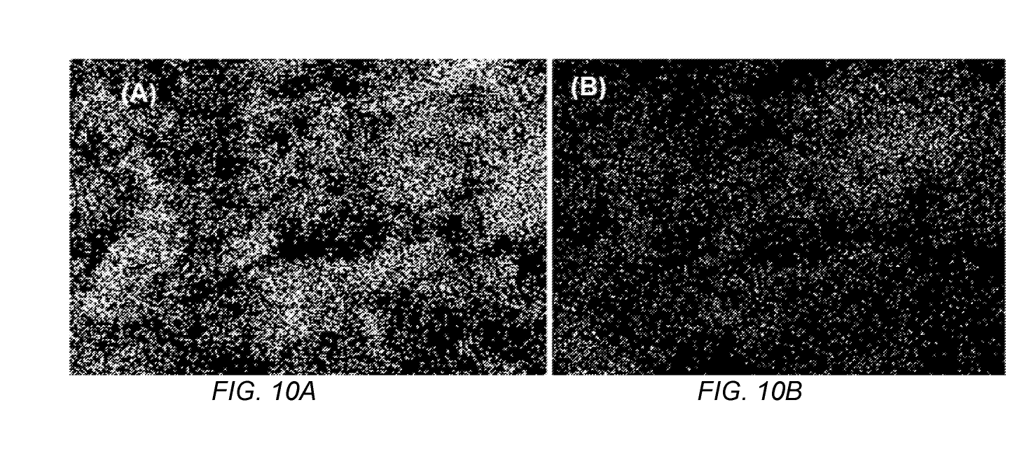

The elemental compositions of MGCN-6-X were investigated by elemental CHN analysis. Elementary analysis was carried out by using a Yanaco MT-5 CHN analyzer. The carbon to nitrogen atomic ratio of the material was found to be 0.73 which was in close agreement with the values obtained from EDAX studies (Energy dispersive X-Ray Analysis) shown in FIG. 9. FIGS. 10A and 10B show the elemental mapping of C and N atoms in the MGCN-6 sample. The data reveal that the carbon (C) and nitrogen (N) were uniformly distributed throughout the sample. No other elements were found in the elemental mapping, indicating that the material was composed of C and N. From analysis of FIGS. 10A and 10B, the MGCN samples had a greater amount of nitrogen content than the carbon content. Moreover, a uniform dispersion of the carbon and nitrogen throughout the samples was visible.

5. FT-IR and UV-Vis

The FTIR spectra were recorded by using Perkin Elmer spectrum 100 series, bench top model equipped with the optical system that gives the data collection over the range of 7800 to 370 cm⁻¹. The spectra were recorded by averaging 200 scans with a resolution of 2 cm⁻¹, measuring in transmission mode using the KBr self-supported pellet technique. The spectrometer chamber was continuously purged with dry air to remove water vapor. UV-Vis absorption spectra of the materials were recorded by using LAMBDA 750 UV/VIS/NIR spectrophotometer (190 nm-3300 nm) from Perkin Elmer. Instrument was equipped with a diffuse reflectance integrating sphere coated with BaSO₄, which serve as a standard. Thickness of the quartz optical cell was 5 mm. The band gaps of the materials were calculated using Tauc Plot method.

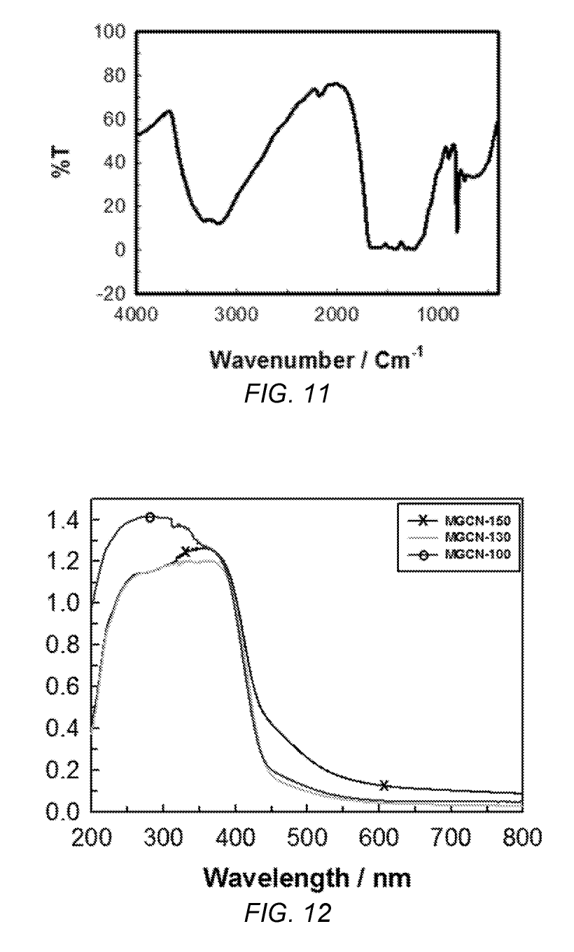

FIG. 11 of Fourier-transform infrared (FT-IR) spectroscopic measurements shows the existence of condensed CN heterocycles, as they exhibits the typical bending mode of CN heterocycles at 800 cm⁻¹ as well as stretching mode of the corresponding rings between 1200 and 1600 cm⁻¹. The broad band appearing in the range of 3000 to 3500 cm⁻¹ results from the uncondensed amino groups present in the structure. This confirms the formation of polymeric melon wall structure in the mesoporous graphitic carbon nitride. Since the product is a porous material the surface was terminated with amino groups (—NH₂) in order to maintain connectivity.

FIG. 12 shows the UV-Vis diffuse reflectance spectrums of MGCN-6-100, MGCN-6-130 and MGCN-150. All of these materials show an absorption pattern of a semiconductor with a pronounced band gap at about 420 nm, thus making the material slightly yellow.

6. XPS

X-ray spectroscopy measurements were carried out using PHI Quantera SXM (ULVAC-PHI) instrument with a 20 kV, Al Kα probe beam (E=1486.6 eV). Prior to the analysis, the samples were evacuated at high vacuum (4×10 Pa) and then introduced into the analysis chamber. For narrow scans, analyzer pass energy of 55 eV with a step of 0.1 eV was applied. To account for the charging effect, all the spectra were referred to the C1s peak at 284.5 eV. Survey and multiregion spectra were recorded at C1s and N1s photoelectron peaks. Each spectral region of photoelectron interest was scanned several times to obtain a good signal-to-noise ratio.

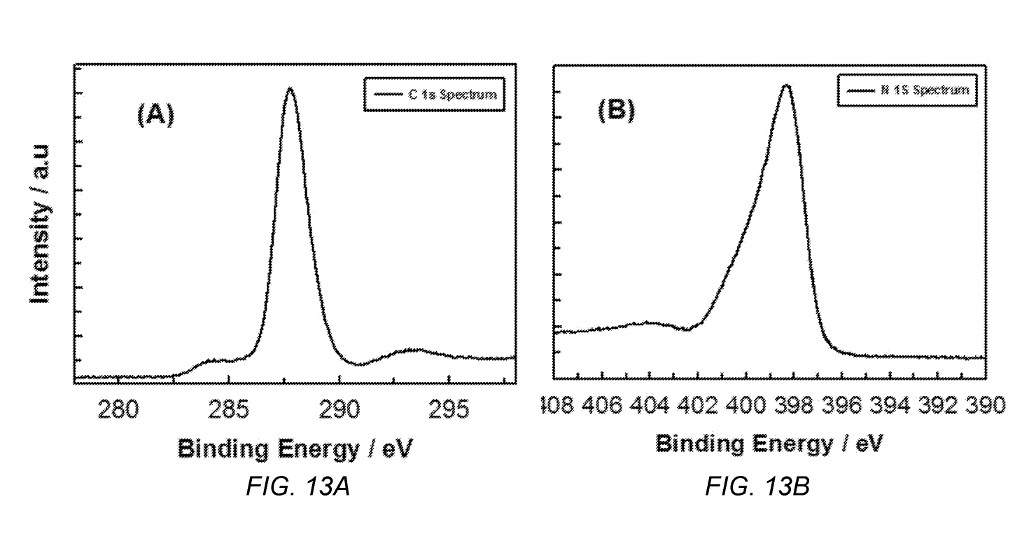

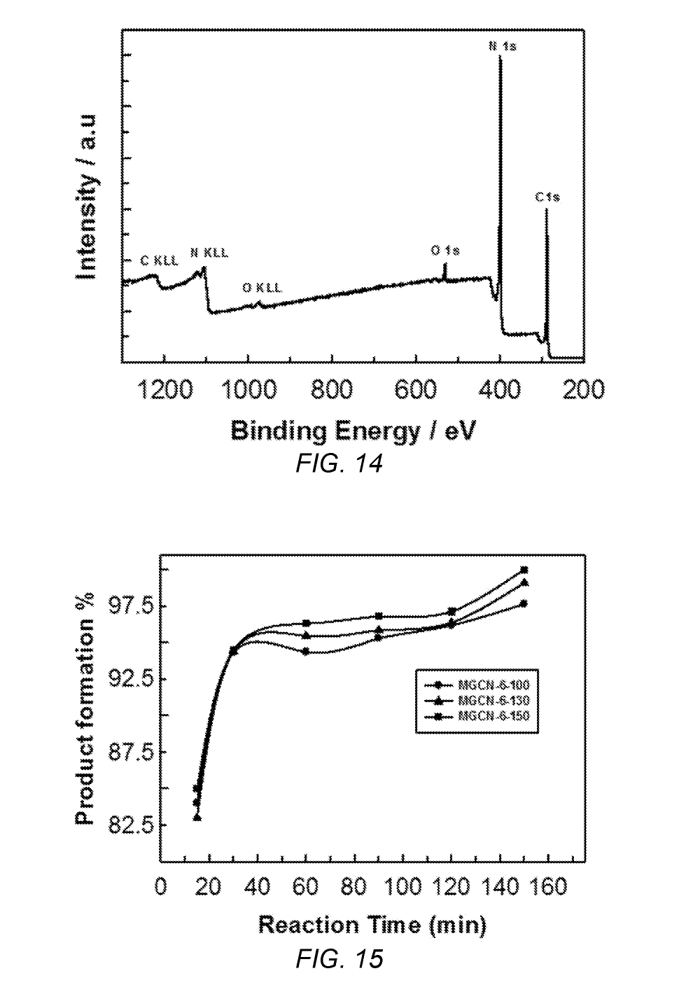

XPS measurements can reveal further details about the mesoporous graphitic carbon nitride polymer. FIG. 13A shows the C is binding energy was mainly a one carbon species with a binding energy of 288.2 eV, corresponding to a C—N—C coordination. FIG. 13B shows the N is spectrum several binding energies can be separated. The main signal shows occurrence of C—N—C groups (398.7 eV) and tertiary nitrogen N—(C)3 groups (400.1 eV) in about the expected ratio. Deconvolution of the XPS signals in FIG. 14 also reveals a weak additional signal at 401.4 eV, indicative of amino functions carrying hydrogen (C—N—H). It is however important to underline that the peak of tertiary amines was stronger than these hydrogen bound amines. This proves a degree of condensation well beyond the linear polymer melon structure.

### Example 4

### Knoevenagel Condensation Using MGCN-6 Materials

The metal-free MGCN-6 materials of Example 2 of the present invention having high surface area and large pore volume were used as a basic catalyst for the Knoevenagel condensation between benzaldehyde (1) and malononitrile (2) in ethanol at room temperature to yield 2-benzylidenemalononitrile (3) according to the scheme below. Benzaldehyde (benzaldehyde (106.1 mg; Mwt: 106.02) and malononitrile (79.3 mg; Mwt: 66.06) at a molar ratio of (1:1.2) was added to ethanol (5 g) at room temperature. The reaction mixture was stirred at room temperature.

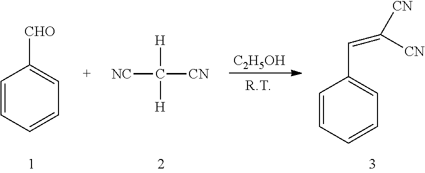 [SMILES: "C2H5OH" ; O=Cc1ccccc1 ; N#CCC#N ; N#CC(C#N)=Cc1ccccc1]

FIG. 15 shows the reaction progress of a Knoevenagel condensation using MGCN-6 materials of the present invention. The MGCN-6 catalysts are highly active and afford high yields in a short time. MGCN-6-150 shows the highest conversion and 100% product selectivity. This was attributed to the high surface area and large pore volume of the catalyst that provided increased numbers of basic sites.

In summary, the graphitic carbon nitrides (MGCN-6) with a three-dimensional Ia3d body centered cubic arrangement of the present invention have tunable pore diameters and high nitrogen content. These materials were successfully fabricated employing KIT-6 mesoporous silica template with different pore diameters prepared by a mixture of Pluronic P-123 triblock copolymer and n-butanol, with cyanamide (NCNH₂) as a precursor. From the analysis of the above-described data, the MGCN of the present invention possess a three dimensional cubic structure with pair of independently interpenetrating three dimensional continuous networks of mesoporous channels that are mutually intertwined and separated by graphitic carbon nitride walls. Moreover, the sample exhibited high surface area, pore volume, and uniform pore size distribution. The performances of MGCN-6 samples of the present invention were tested in the base-catalyzed Knoevenagel condensation. The catalyst was highly active and afforded a high yield of the corresponding product in a short reaction time. The catalyst was also highly stable and could be recycled. Recycling was done by the catalyst that was filtered from reaction mixture and activated at 200° C. in air to ensure it free from the reactants and products. The methods described herein of tuning the pore diameter of the mesoporous silica template to control the textural parameters, especially the pore diameter, and the high nitrogen content of the mesoporous carbon nitride materials offer a unique pathway for fabricating porous nanostructured nitrides with very high nitrogen contents and tunable textural parameters.

1. A mesoporous graphitic carbon nitride material (MGCN) and comprising sheets of three dimensionally arranged s-heptazine units, and having a pore volume between 0.70 and 0.80 cm³ g⁻¹, a surface area of 275 to 300 m² g⁻¹, and an atomic carbon to nitrogen ratio of 0.7 to 0.8, wherein the MGCN material is derived from cyanamide and the cyanamide is templated with a hard KIT-6 template.

2. The mesoporous material of claim 1, wherein the material has a d spacing of 89 to 92.

3. (canceled)

4. The mesoporous material of claim 1, wherein the material has a pore volume between 0.75 and 0.77 cm³ g⁻¹, and a surface area of 275 to 285 m² g⁻¹, and an atomic carbon to nitrogen ratio of 0.7 to 0.8.

5. The mesoporous material of claim 4, having a pore diameter of 4 to 4.2 nm.

6. The mesoporous material of claim 1, wherein the material has a pore volume between 0.769 cm³ g⁻¹, and a surface area of 280.5 m² g⁻¹, an atomic carbon to nitrogen ratio of 0.7 to 0.8, and a pore diameter of 4.2 nm.

7. A condensation reaction process comprising:
(a) contacting the mesoporous graphitic carbon nitride material of claim 1 with a carbonyl containing compound and an activated methylene containing compound forming a reactant mixture; and
(b) subjecting the reactant mixture to conditions suitable to condense the carbonyl and methylene group to form a carbon-carbon bond, wherein the conditions include a temperature of 10 to 30° C.

8. (canceled)

9. The process of claim 7, wherein the mesoporous graphitic carbon nitride material has a pore volume between 0.769 cm³ g⁻¹, and a surface area of 280.5, an atomic carbon to nitrogen ratio of 0.7 to 0.8, and a pore diameter of 4.2, and the aldehyde is benzaldehyde and the activate methylene containing compound is malononitrile, and 2-benzylidenemalononitrile is produced in a yield of at least 92%.

10. The process of claim 9, wherein the benzylidenemalononitrile selectivity is at least 98%.

11. The process of claim 7, wherein the condensation process is a Knoevenagel reaction.

12. A method of producing a mesoporous graphitic carbon nitride material of claim 1, the method comprising:
(a) mixing a calcined hard KIT-6 template with an aqueous cyanamide solution forming a hard template reactant mixture;
(b) subjecting the hard KIT-6 template reactant mixture to conditions suitable to form a templated carbon nitride composite;
(c) heating treating the KIT-6 templated carbon nitride composite to a temperature of 450 to 550° C. to form a mesoporous graphitic carbon nitride material/template complex wherein the heating step (c) is performed under a nitrogen flow; and
(d) removing template from the mesoporous graphitic carbon nitride material/KIT-6 template complex producing graphitic carbon nitride material comprising sheets of three dimensionally arranged s-heptazine units.

13. (canceled)

14. The method of claim 12, wherein the aqueous cyanamide solution is 40-60% cyanamide by weight.

15. The method of claim 12, wherein the aqueous cyanamide solution is 50% cyanamide by weight.

16. The method claim 12, wherein the heating step (c) is at a temperature of 500° C.

17. (canceled)

18. The method of claim 15, wherein the nitrogen flow is at 50 mL per minute.

19. The method of claim 18, wherein the temperature of heating step (c) is achieved using a heating rate of about 2.0° C. per minute.

20. (canceled)
